(19) United States
(12) Patent Application Publication
(10) Pub. No.: US 2006/0142848 A1
(43) Pub. Date: Jun. 29, 2006

(54) Extra-anatomic aortic valve placement

(76) Inventor: Shlomo Gabbay, New York, NY (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/355,732

(22) Filed: Feb. 16, 2006

Related U.S. Application Data
(63) Continuation-in-part of application No. 10/266,380, filed on Oct. 8, 2002.
Application No. 10/266,380 is a continuation-in-part of application No. 09/973,609, filed on Oct. 9, 2001.
Application No. 09/973,609 is a continuation-in-part of application No. 09/659,882, filed on Sep. 12, 2000, now abandoned.

(51) Int. Cl. A61F 2/06; A61F 2/24

(52) U.S. Cl. 623/1.26; 604/9; 623/2.11; 623/2.14

(57) ABSTRACT

A method for extra-anatomic valve placement includes creating an aperture through the patient's heart that extends from a location outside of the patient's heart and into a left ventricle of the heart. A valve is mounted at least partially within the aperture and a continuous path is provided for fluid communication from the mounted valve and into the patient's aorta. The method can be implemented in the absence of cardio pulmonary bypass.

### CROSS-REFERENCE TO RELATED APPLICATIONS

This application is a continuation-in-part of application Ser. No. 10/266,380, which was filed on Oct. 8, 2002, and entitled HEART VALVE PROSTHESIS AND SUTURELESS IMPLANTATION OF A HEART VALVE, which is a continuation-in-part of U.S. patent application Ser. No. 09/973,609, which was filed on Oct. 9, 2001, and entitled HEART VALVE PROSTHESIS AND SUTURELESS IMPLANTATION OF A HEART VALVE PROSTHESIS, which is a continuation-in-part of U.S. patent application Ser. No. 09/659,882, which was filed on Sep. 12, 2000 and entitled VALVULAR PROSTHESIS AND METHOD OF USING SAME, all of which applications are incorporated herein by reference.

### TECHNICAL FIELD

The present invention relates generally to an extra-anatomic aortic valve placement, which can be employed to replace the function of a patient's aortic valve.

### BACKGROUND

A heart valve can become defective or damaged, such as resulting from congenital malformation, disease, or aging. When the valve becomes defective or damaged, the leaflets may not function properly. One common problem associated with a degenerating heart valve is an enlargement of the valve annulus (e.g., dilation). Other problems that may result in valve dysfunction include chordal elongation, lesions developing on one or more of the leaflets and calcification of the valve.

It is well known to utilize mechanical heart valves, such as the ball check valve, and natural tissue cardiac valves to replace defective aortic and mitral valves in human patients. One type of natural tissue heart valve typically employs a porcine valve for implantation in a human, as they are very similar to human valves of appropriate size and generally are easy to procure. Typically, the porcine valve is fixed by chemically treating it, such as with an appropriate glutaraldehyde solution. The treated porcine valve further may be mounted into a stent to support the valve at a fixed position.

A stent typically is formed of a resilient material, such as a plastic (e.g., DELRIN). Examples of various stent structures are disclosed in U.S. Pat. No. 3,983,581, U.S. Pat. No. 4,035,849. The stent usually is covered with a fabric material, such as DACRON or a suitable textile material. The fabric material provides structure for securing the valve relative to the stent. The stented heart valve prosthesis may be implanted into a patient for a heart valve replacement.

In order to surgically implant a heart valve into a patient, the patient typically is placed on cardiopulmonary bypass during a complicated, but common, open-chest and open-heart procedure. In certain situations, an individual requiring a heart valve replacement may be sufficiently ill, such that placing the individual on cardiopulmonary bypass may pose too great of risk. As one example, such individuals may correspond to a class of patients who may have severe aortic valve insufficiency. Patients with aortic valve defects often may also exhibit calcification of the aortic valve and the aorta, including one or both of the aortic arch and the descending aorta. When the aorta is calcified, there are increased risks associated with performing cardio pulmonary bypass, as is typically performed for aortic valve replacement procedures. Additionally, patients having a diseased or defective aortic valve may be too ill to survive conventional open-heart surgery, which may include cardio pulmonary bypass.

Patients exhibiting these and other conditions in the aortic valve would benefit from a low invasive approach for replacing the function of the aortic valve.

### SUMMARY

The present invention relates generally to an extra-anatomic aortic valve placement, which can be employed to replace the function of a patient's aortic valve.

One aspect of the present invention provides an extra-anatomic aortic valve placement method. The method includes creating an aperture through the patient's heart that extends from a location outside of the patient's heart and into a left ventricle of the heart. A valve is mounted at least partially within the aperture and a continuous path is provided for fluid communication from the mounted valve and into the patient's aorta. The method can be implemented in the absence of cardio pulmonary bypass.

Another aspect of the present invention provides a method for extra-anatomic aortic valve placement. The method includes creating an aperture through the patient's heart that extends from a location outside of the patient's heart and into a left ventricle of the heart (e.g., through the apex of the patient's heart). The aperture is obstructed to inhibit blood loss through the aperture. A valve is mounted at least partially within the aperture while the aperture is obstructed. A length of a flexible conduit is attached between an outflow end of the valve and the patient's aorta to provide for substantially unidirectional flow of blood from the left ventricle, through the conduit and into the patient's aorta, the method being performed in the absence of cardio pulmonary bypass.

Still another aspect of the present invention provides a system for extra-anatomic aortic valve placement. The system includes means for creating an aperture through the patient's heart that extends from a location outside of the patient's heart and into a left ventricle of the heart. The system also includes means for obstructing the aperture to inhibit blood loss through the aperture and valve means for, when mounted at least partially within the aperture, providing substantially unidirectional flow of blood from the left ventricle and through the means for providing. The system also includes means for, when connected between the valve means and the patient's aorta, providing for fluid communication along a continuous path from the left ventricle into the patient's aorta.

### BRIEF DESCRIPTION OF THE DRAWINGS

FIG. 1 depicts an example of a system that can be utilized for extra-anatomic aortic valve placement according to aspect of the present invention.

FIG. 2 depicts part of an extra-anatomic aortic valve placement procedure being implemented according to aspect of the present invention.

FIG. 3 depicts another part of an extra-anatomic aortic valve placement procedure being implemented according to aspect of the present invention.

FIG. 4 depicts yet another part of an extra-anatomic aortic valve placement procedure being implemented according to aspect of the present invention.

FIG. 5 depicts a valve mounted to the heart as part of an extra-anatomic aortic valve placement procedure being implemented according to aspect of the present invention.

FIG. 6 depicts another part of an extra-anatomic aortic valve placement bypass procedure being implemented according to aspect of the present invention.

FIG. 7 depicts a completed extra-anatomic aortic valve placement according to aspect of the present invention.

### DETAILED DESCRIPTION

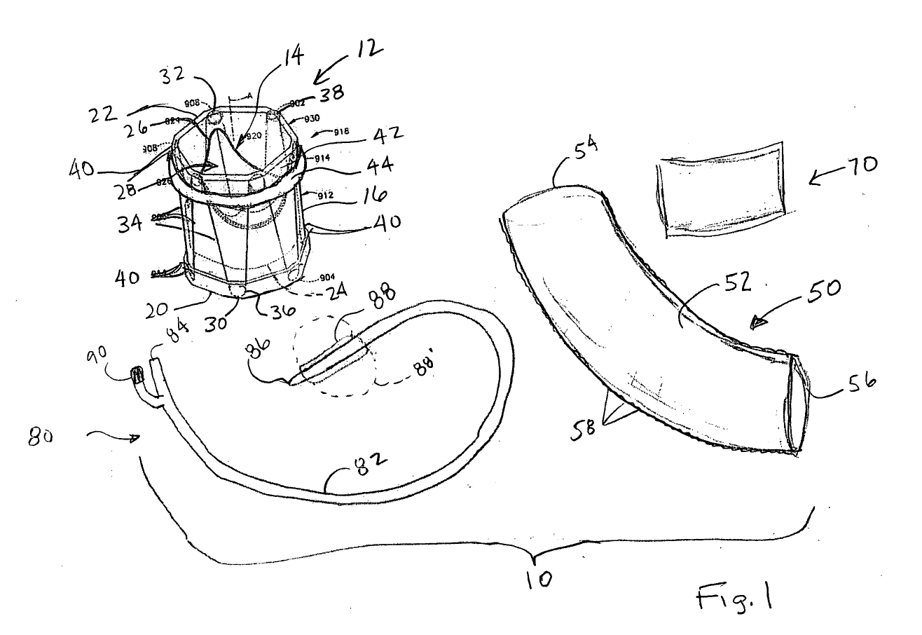

FIG. 1 depicts a system that can be utilized as part of an extra-anatomic aortic valve placement procedure implemented according to aspect of the present invention. The system of FIG. 1 enables an extra-anatomic aortic valve placement procedure to be implemented in the absence of cardio pulmonary bypass. The method can also be implemented in a minimally invasive manner, such as described herein. Thus, the procedure can be implemented with reduced risk to the patient relative to existing procedures. Additionally, the approach described herein makes it feasible to perform the procedure and effectively replace the function of the patient's aortic valve in circumstances (e.g., when the aorta is severely calcified) where traditional aortic valve replacement may not have a viable option.

In the example of FIG. 1, the heart valve prosthesis 12 includes a valve member 14 mounted within a support 16. The support 16 can be of a type that can expand from a reduced cross-sectional condition to an expanded condition. The expansion can occur automatically (e.g., due to the support being self-expanding). Alternatively or additionally, the expansion can be assisted, such as by use of a balloon catheter or a mechanical device that urges the support radially outwardly from its reduced cross-sectional condition.

The valve member 14 can be mounted within the support between an inflow end 20 and an outflow end 22 of the prosthesis 12. In FIG. 1, an inflow end 24 of the valve member 14 can be positioned adjacent the inflow end 20 and the outflow end 26 of the valve member 14 is positioned near the outflow end of the prosthesis. The valve member 14 can be natural tissue valve, such as a homograft or xenograft valve, or it can be manufactured from one or more sheets of biocompatible material. As described herein, the valve member 14 can also be a mechanical valve or a biomechanical valve. The valve member 14 can include sidewall portion, which can be a tubular valve wall, such as for a homograft or xenograft.

The valve member 14 includes at least one moveable member 28 that is configured as means for providing substantially unidirectional flow of blood through the valve prosthesis 12. In the example of FIG. 1, the moveable member 28 of the natural tissue valve member 14 includes a plurality of leaflets that extend radially inwardly from a sidewall portion of the valve. The leaflets coapt along their adjacent edges to provide for substantially unidirectional flow of blood through the valve prosthesis 12. The outflow end 26 of the valve prosthesis 12 can have a generally sinusoidal contour, as shown in FIG. 1, although it is not limited to such an outflow contour. The valve member 14 can be connected within the support 16 via sutures or other known connecting means (e.g., clips, staples or the like), for example.

The support 16 includes axially spaced apart ends 30 and 32 interconnected by generally axially extending support features 34. In the example of FIG. 1, adjacent support features 34 are interconnected by arcuate junctures 36 and 38 at the respective ends 30 and 32 so as to define a generally sinusoidal (or zig-zagging) sidewall portion arranged in a generally cylindrical configuration. In the example of FIG. 1, there are six junctures 36 and 38 at each of the respective ends 30 and 32 that are interconnected by associated axially extending support features 34. Those skilled in the art will understand and appreciate that other numbers (e.g., 2, 3, 9, 12 and so forth) and configurations of end junctures 36 and 38 can be utilized in accordance with an aspect of the present invention. For example, as an alternative to curved interconnecting end junctures 36, 38 shown in the FIG, such ends could be pointed or rectangular, hexagonal or the like. The arcuate junctures 36 and 38 can be mechanically biased to urge each adjacent pair of the support features 34 apart so as to cause the prosthesis 12 to return to its expanded condition.

The support 16 further includes one or more projections or spikes 40 that extend axially and radially outwardly from at least some of the respective end junctures 36 and/or 38 of the support. While a pair of such spikes 40 are illustrated as associated with each end juncture 36, 38, other number of spikes can be implemented, such as single spike or more than two spikes at some or all of the junctures. In the example illustrated in FIG. 1 the pairs of spikes at opposite ends operate to mitigate movement in different directions, such as by having each spike 40 forming an acute angle relative to its associated axial support feature 34 adjacent to which it extends. Additional spikes may also extend radially outwardly form the support features 34.

The support 16 can be formed a shape memory material, such as NITINOL. For example, the support can be formed from a small cylindrical tube of the shape memory material, such as via a laser cutting (ablation) process in which the desired sinusoidal sidewall is cut from the tube. In this way, the support features 34, the interconnecting end junctures 36 and 38, and associated spikes 40 can be formed as an integrated (e.g., monolithic) structure having a desired shape and size. Additionally, ends of the spikes 40 can have tapered or sharpened tips to facilitate gripping surrounding tissue when implanted. For example, the spikes 40 can be formed by laser cutting from the same tube or, alternatively, they could be welded onto the support 16 at desired positions. The resulting structure can then be heated to its transformation temperature and forced to a desired cross-sectional dimension and configuration (its austentic form), such as shown in FIG. 1. The support 16 can then be bent or deformed to a reduced cross-sectional dimension when in its low-temperature (martensitic) form to facilitate its mounting within a barrel of an implanter, for example. The arcuate junctures 36 and 38 can also be mechanically biased to urge the associated pair of support features apart from each other so as to urge prosthesis 12 to its expanded condition.

The prosthesis 12 can also include an outer sheath 42 of a substantially biocompatible material. The outer sheath 42 covers at least a substantial amount of exposed portions of the support 16, such as including the ends 20 and 22, to mitigate contact between the blood and the support when the prosthesis 12 is implanted. The valve member 14 further can be attached relative to the sheath 42, such as by sutures along the inflow and outflow ends of the prosthesis. Such sutures (not shown) further can connect the valve member 14 and the sheath 42 relative to the support 16. The outer sheath 42 can cover the entire support, such that all non-biological material is completely covered, for example. The outer sheath 42 can be formed of one or more natural tissue sheets (e.g., animal pericardium, dura matter, fascia lata), although other natural or synthetic biocompatible materials also could be used to provide an outer sheath in accordance with an aspect of the present invention.

The natural tissue material utilized to provide the outer sheath 42 can include a NO-REACT® tissue product, which is commercially available from Shelhigh, Inc., of Union, N.J. The NO-REACT® tissue products help improve the biocompatibility of the apparatus 50, thereby mitigating the likelihood of a patient rejecting an implanted prosthesis that includes the apparatus. The NO-REACT® tissue products also has been shown to resist calcification when implanted in vivo. The NO-REACT® tissue products further have been shown to facilitate growth of endothelium after being implanted.

The prosthesis 12 can also include an outflow flange 44 that extends radially outwardly from the sidewall of the prosthesis adjacent the outflow end 22. The flange 44 facilitates attachment to the heart, such as described herein. The flange 44 can be any substantially biocompatible material, such as a natural tissue material (e.g., pericardium, dura matter, fascia lata), a synthetic material (e.g., DACRON) or a combination of natural and synthetic materials (e.g., a collagen impregnated fabric). One example of a natural tissue is a sheet of animal pericardium that has been fixed and substantially detoxified, such as formed from NO-REACT tissue product mentioned herein. The flange 44 can be attached to the exterior of the prosthesis 12 (e.g., by sutures). For example, the flange 44 can be spaced apart from the outflow end 22 a small distance, such as from about 2 mm to about 7 mm, which distance may vary depending on the size of the valve prosthesis 12 and/or the size of the patient's heart. The flange 44 can extend radially outwardly from the sidewall of the prosthesis 12 a predetermined distance (e.g., from about 3 mm to about 10 mm).

While the example valve 12 in FIG. 1 is depicted as a self-expanding, natural tissue heart valve prosthesis 12, those skilled in the art will understand and appreciate that the system 10 and the approach described herein are not limited to use of such a valve. For example, the system 10 can be implemented with a mechanical heart valve, as well as other natural tissue heart valves. Additionally, or alternatively, the valve prosthesis 12 need not be self-expanding, as it may be expanded manually such as by a balloon or other mechanical device located within the valve. As yet another alternative, the valve prosthesis 12 may be a non-expandable type of valve (e.g., mechanical, natural tissue).

The system 10 also includes an elongated flexible conduit 50. The conduit 50 includes a side wall portion 52 that extends longitudinally between spaced apart ends 54 and 56. The conduit can be curved to facilitate attachment between the heart and a patient's aorta, such as described herein. At least one of the ends 54, 56 is dimensioned and configured for attachment to the outflow end of the valve prosthesis 12. For example, the end 54 can be attached (e.g., by sutures) in a circumscribing relationship with the outflow end portion 22 of the valve prosthesis 12 such that the combined valve and conduit 50 provides for substantially unidirectional flow of fluid therethrough.

As an example, the conduit 50 is formed of a tube of a substantially biocompatible material and is configured to provide for fluid communication between the spaced apart ends 54 and 56. For instance, the conduit 50 can be formed of a biological material (e.g., animal pericardium, dura matter, collagen) or synthetic material (e.g., DACRON, another polymer or the like) or as well as a combination of materials which can be natural and/or synthetic. By way of further example, the conduit 50 can be formed from an elongated sheet of animal pericardium that can be folded along a longitudinal axis and its corresponding side edges attached together and in which the tubular portion is fixed over a substantially corrugated mandrel to provide circumferential corrugations 58 along its length. Those skilled in the art will understand and appreciate that the corrugations 58 can be provided in other ways for the conduit 50.

The system 10 can also include a sheet of a biocompatible flexible material that can be applied to the heart to facilitate attachment of the valve 12 and the conduit 50 to the patient's heart. The sheet 70 can be substantially rectangular sheet as shown in FIG. 1, although other shapes and configurations of sheets can be utilized. The sheet 70 can be formed of a NO-REACT® tissue product, such as described herein. The sheet 70 is dimensioned and configured to provide a surface that is larger in diameter than the outflow end of the valve 12. In this way, corresponding aperture can be formed through the sheet 70 sufficient to permit the outflow end portion of the valve to extend therethrough when implanted.

The system 10 also includes a balloon catheter 80 includes an elongated tube 82 of a substantially flexible material that extends between a proximal end 84 and a distal end 86. A balloon circumscribes a portion of the tubular member 82 near the distal end 86. The balloon 88 is in fluid communication with a lumen that extends longitudinally through the tubular member 82, which can be utilized to inflate the balloon 88 to an expanded condition, indicated by dashed lines at 88′. As an example, the lumen within the tubular member 82 is connected to an inflation member 90 that is coupled to the tubular member 82 near the proximal end 84. The inflation member 90 provides access to the lumen for inflation and deflation of the balloon 88. As an example, an inflation fluid such as air, saline, plasma, blood or other biocompatible inflation fluid can be introduced into the lumen of the tubular member 82 via the inflation member 90. The balloon 88 thus responds to introduction of inflation fluid by inflating toward its expanded condition 88 commensurate with the amount of fluid introduced into the catheter 80. As an example, the inflation fluid can be introduced by a syringe or other inflation mechanisms known or yet to be developed in the art.

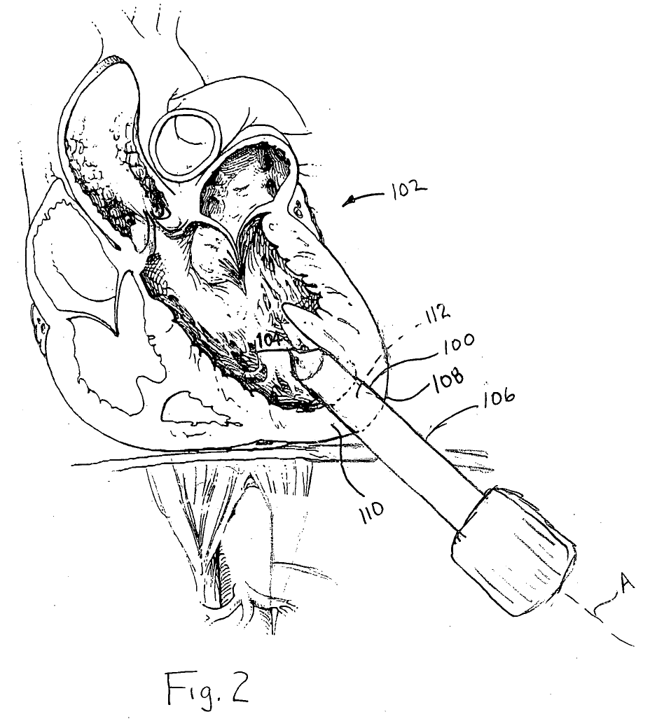

The system 10, or at least a portion thereof, can be employed to perform an extra-anatomic aortic valve placement procedure according to aspect of the present invention. FIGS. 2 through 7 depict one example of a procedure that can be implemented in the absence of cardio pulmonary bypass (CPB) according to aspect of the present invention. Because the procedure can be used without CPB, the procedure is well-suited for traditionally high risk patients. Additionally, the procedure can be formed without cutting open the heart as is also typically performed in an aortic valve replacement procedure. As shown and described herein, the extra-anatomic aortic valve placement is performed by providing an extra-anatomic valve and conduit between the patient's left ventricle and the aorta, such as the descending aorta.

FIG. 2 depicts part of a procedure in which an aperture 100 is being formed in a patient's heart 102. In the example of FIG. 2, the aperture 100 is formed by cutting a hole that extends through the patient's heart muscle and into the left ventricle 104. As an example, a sharpened hollow elongated, cylindrical cutting implement 106 can be inserted into the apex 108 of the heart 102 through the heart muscle 110 and into the left ventricle 104. The cutting implement 106 can be rotated about a longitudinal axis A to facilitate its insertion through the muscle tissue 110 so that the corresponding section of the heart muscle remains within the cutting implement 106. That is, upon removal of the cutting implement 106 from the patient's heart 102, the corresponding section of the heart muscle 112 remains within the interior of the cutting implement 106, such that a corresponding aperture 100 is provided in the apex 108 of the patient's heart 102. The aperture 100 provides an open passage from a location exterior to the patient's heart into the left ventricle 104. Those skilled in the art will understand and appreciate that other types of cutting implements, including scalpels, knives and the like, can be utilized to form the aperture 100 in the patient's heart 102. The aperture 100 is dimensioned and configured to have a diameter that approximates, or is slightly smaller than (e.g., about 2 to 10% smaller than), the outer diameter of the valve 12, such as when in its fully expanded condition. Thus, in the example of FIG. 2, the inner diameter of the cylindrical cutting implement 106 is selected according to the size of the heart valve prosthesis 12 to be implanted.

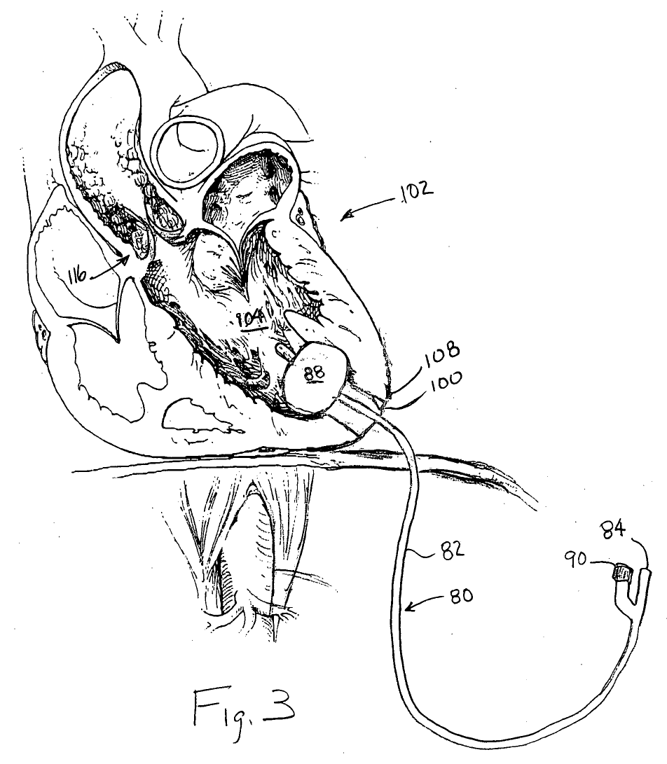

FIG. 3 depicts another part of the procedure in which the aperture 100 is obstructed to mitigate blood flow or loss through the aperture. In the example of FIG. 3, a balloon catheter 80 is inserted through the aperture 100 so that the balloon 88 thereof resides within the left ventricle 104. The balloon 88 is inflated, such as by introduction of an inflation fluid through the inflation member 90 to inflate the balloon to an expanded condition. Thus, in the example of FIG. 3, a proximate surface of the inflated balloon 88 engages a ventricle peripheral portion of the aperture 100 thereby substantially sealing the aperture. Prior to the insertion of the catheter within the patient's heart 102, a surgeon's thumb or other implement may be used to plug the aperture 100 from the exterior until the catheter is inserted and the balloon 88 is inflated. In the example of FIG. 3, as described herein, the catheter 80 can be a Foley catheter, although other types of catheters can be utilized to obstruct the aperture to mitigate blood loss from the heart. Because the catheter 80 is utilized in this way, cardio pulmonary bypass is not required. Consequently, the patient's heart 102 may continue pumping blood to the patient's body through the patient's aortic valve 116 until the procedure is substantially complete. Advantageously, the catheter 80 can remain in the patient's heart 102 during a significant portion of the procedure, thereby obviating the need for cardio pulmonary bypass.

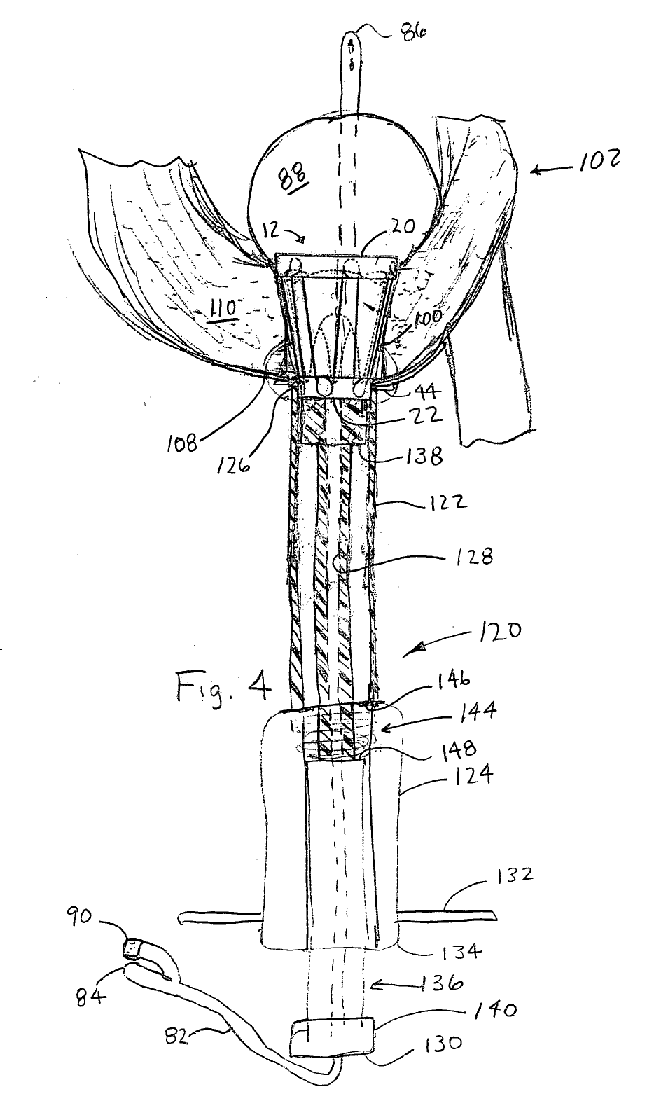

FIG. 4 depicts another part of the procedure in which the valve 12 is being implanted at least partially within the aperture 100 of the patient's heart 102. Additional information about the particular heart valve 12 being utilized in the example of FIG. 4 can be made with reference back to FIG. 1. In the example of FIG. 4, the valve 12 is being discharged from an implanter apparatus 120. The valve 12 is arranged so that the inflow end 20 is located adjacent the ventricular peripheral portion of the aperture 100 interior to the heart 102 and the outflow end 22 is located adjacent the apex 108 exteriorized relative to the patient's heart. In the example of FIG. 4, the outflow end portion 22 of the prosthesis 12 can extend outwardly from the patient's heart a distance (e.g., about 2 mm to about 7 mm) outwardly from the apex 108. This is approximately the same distance that the flange 44 of tissue is spaced apart from the outflow end of the valve 12 as to enable its attachment to the patent's heart circumscribing the aperture 100.

As shown in FIG. 4, the elongated tubular member 82 of the catheter 80 extends axially through the prosthesis 12 (e.g., between the valve leaflets) and through the corresponding lumen 128 that extends axially through the implanter apparatus 120. Because the catheter remains in place while the prosthesis 12 is discharged from the barrel 122 of the implanter apparatus 120, there is little, (if any) loss of blood from the patient's heart during the implantation procedure. Thus, cardio pulmonary bypass is not necessary during the implantation and attachment of the valve 12 within the aperture 100 formed in the patient's heart 102.

By further example, the implanter 120 can be employed to facilitate sutureless implantation of the valve 12 into the aperture 100, such as under direct vision of the surgeon into the aperture 100. The implanter 120 includes an elongated cylindrical barrel 122 that extends from a body portion 124 and terminates in a distal open end 126. The barrel 122 has an inner diameter that is less than the outer diameter of the valve prosthesis 12 in its expanded condition. Thus, in order to insert the prosthesis 12 into the barrel 122, the prosthesis is deformed to a reduced cross-sectional dimension, that is less that its fully expanded condition.

For example, the inner diameter of the barrel 122 can range from about 5 mm to about 20 mm, whereas the outer diameter of the valve prosthesis 12 (in its expanded condition) typically ranges from about 15 mm to about 35 mm. Thus, the barrel 122 can accommodate a prosthesis 12, which has been deformed to reduced cross-sectional dimension, without compromising the durability and operation of the valve. The exterior of the barrel 122 further can include indicia (e.g., ruler markings) 710 that can help indicate the distance the barrel is inserted into a patient's heart 102, such as for positioning the inflow end 20 of the prosthesis 12 a predetermined distance into the heart.

By way of further example, prior to reducing the cross-sectional size of the valve and before inserting the prosthesis 12 into the barrel, the elongated tubular member 82 of the catheter 80 can be inserted axially through the valve member 28 of the prosthesis 12. The prosthesis 12 can then be slid along the tubular member 82 toward the aperture 100 so that the remaining length of the tubular member 82 and the proximal end extend therefrom. The tubular member 82 can then be inserted through a central lumen 128 of the implanter 120 and out a proximal end 130 of the implanter. The lumen 128 also provides a passage through which one or more other elongated objects (e.g., sutures or other instruments) can be inserted. The prosthesis 12 can then be inserted into the barrel 122 of the implanter. In this way, the prosthesis 12 can be implanted without removing the balloon 88 so that cardiopulmonary bypass is not required. Alternatively, the catheter 80 can be inserted through the implanter 120 prior to its insertion and inflation of the balloon within the patient's heart 102.

The implanter 120 also includes a handle 132 that extends outwardly from a proximal end 134 of the body portion 124. A plunger 136 has a distal end 138 that can be urged into engagement with the outflow end 22 of the prosthesis 12 to push the prosthesis discharge it from the open end 126 of the barrel 122. The plunger 136 includes an elongated portion that extends from its distal end 138 and terminates in a proximal end portion 140. The plunger 136 includes the lumen 128 that is sized to enable feeding the tubular member 82 through the implanter 120. The proximal end portion 140 of the plunger 136 operates as a trigger that can be grasped in conjunction with the handle 132 by a surgeon to move the plunger axially through the barrel 122. Other means to discharge the heart valve prosthesis 12 could also be utilized in accordance with an aspect of the present invention. Fluid, such as saline, can also be introduced into the barrel 122, such as through the lumen 128, to facilitate the discharge of the prosthesis 12 from the barrel.

The implanter apparatus 120 can also include a spring (or other means) 144 for resisting movement of the plunger 136 relative to the body 124. The spring 144 engages a distal end 146 within the interior of the body portion 124 and an adjacent shoulder surface 148 of the plunger 136. The spring 144 is thus biased to urge the plunger surface 148 axially apart away from the end 146 of the body portion 124. The amount of tension provided by the spring 144 can be tuned to provide an ergonomic feel for the user.

As mentioned above, the prosthesis 12 includes a self-expanding support 16. Thus, the heart valve prosthesis 12 can expands toward its fully expanded condition as it is discharged from the barrel 122. Alternatively, other means can be employed to expand the valve prosthesis 12. For the example valve 12 shown and described herein, the projections or spikes 40 can insert into surrounding tissue to maintain the valve at a desired axial and angular, thereby anchoring the prosthesis 12 relative to the aperture 100. As the prosthesis 12 is being discharged, the implanter barrel 122 can be concurrently withdrawn from the heart, as is being shown in FIG. 4. It will be understood that different types of valves may be implanted using different methods from that shown in FIG. 4 without departing from the scope of the present invention. The implantation of the valve 12 thus can be customized and modified according to the type of valve being implanted. From the foregoing, the heart valve prosthesis can be mounted in the patient's heart 102 in a minimally invasive manner (e.g., being discharged from the barrel 122 of the implanter 120).

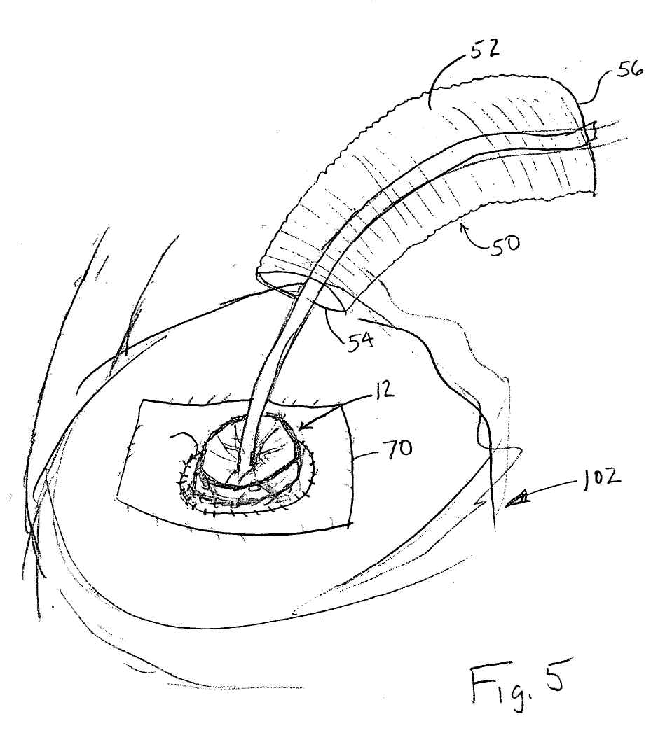

FIG. 5 depicts another intermediate part of the procedure after the valve 12 has been implanted at the aperture 100, such as according to the implantation procedure shown in FIG. 4. Thus, after discharging the prosthesis 12 from the barrel 122 of the implanter apparatus 120, the implanter is withdrawn from the catheter 80. The catheter 80, however, can remain within the heart to inhibit blood flow from the ventricle into the valve 12. In FIG. 5, the valve 12 has been mounted at least partially within the aperture 100 so as to provide a means for providing substantially for unidirectional flow of blood from the ventricle 104 and through the valve 12.

While the implantation can be performed completely sutureless, those skilled in the art will understand and appreciate that one or more sutures can be utilized to further help secure the prosthesis 12 relative to the surrounding tissue 110 along the aperture 100. As depicted in FIG. 5, the outwardly extending flange 44 of the prosthesis 12 is secured overlying the sheet 70 of material. The sheet 70 provides reinforcement for securing the valve 12 to the patient's heart muscle and helps hold the remaining portions of the patient's pericardium over the heart 102. The attachment of the flange 44 to the heart provides additional reinforcement for the valve 12 to help secure the valve relative to the aperture 100 and the patient's heart 102. Those skilled in the art will understand and appreciate other ways that the prosthesis 12 can be appropriately secured to the heart, which may vary according to the type of valve being implanted at the aperture 100.

In FIG. 5, the conduit 50 is shown as being urged over the elongated tubular member 82 of the catheter 80 for subsequent attachment to the outwardly extending portion of the valve 12 and/or to the patient's heart 102. The particular attachment location on the patient's aorta can vary from patient to patient. For example, a significant portion of a patient's aorta may be substantially calcified such that the outflow end 56 of the conduit 50 can be extended to a non-calcified portion to provide an appropriate attachment site. It is to be understood and appreciated that the axial length of the conduit 50 can be longer than required for connecting the patient's heart with an appropriate location on the patient's aorta. An excess length of the conduit 50 thus can be removed (e.g., by cutting) the conduit to a desired length.

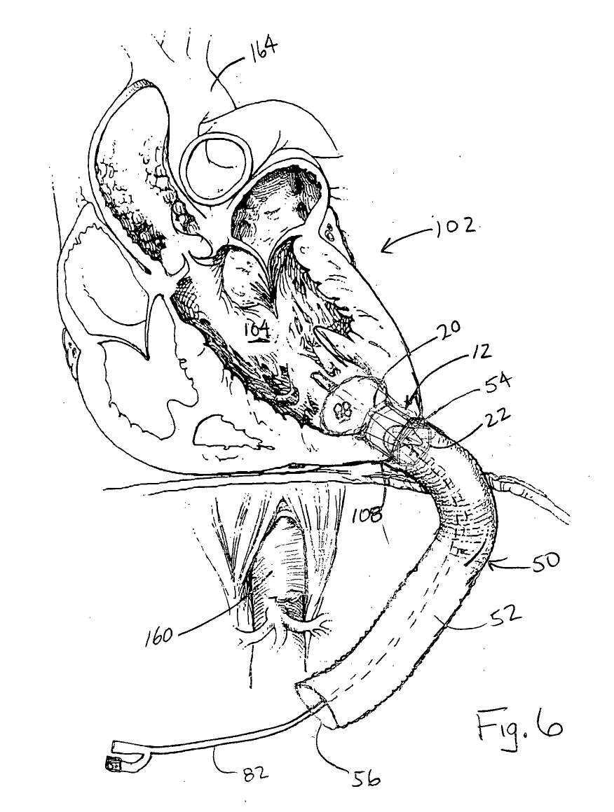

FIG. 6 depicts the conduit 50 attached to the outflow portion of the valve 12 according to the aspect of the present invention. As mentioned herein, the end 54 of the conduit 50 can be anastomosed to the outflow portion of the valve 12, such as by sutures. For instance, the end portion 54 of the conduit 50 can be urged over the radially extended surface of the outflow portion of the valve 12 and attached in a circumscribing relationship around the outflow portion 22. Additionally or alternatively, the conduit 50 can be attached to the heart 102 in a circumscribing relationship relative to the outflow portion of the valve 12. As depicted in FIG. 6, the balloon 88 of the catheter 80 remains obstructing the aperture 100 and the inflow end 20 as the conduit 50 is attached to the valve 12 and/or the heart 102.

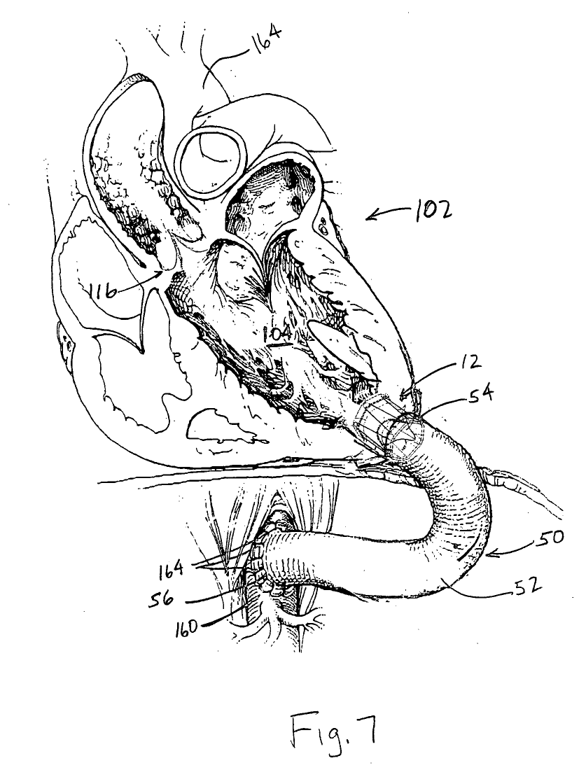

In the example of FIGS. 6 and 7, the free end 56 of the conduit 50, which may be provided by cutting the conduit to a reduced length, is to be attached to the descending aorta 160. It is to be understood and appreciated, however, that the end 56 of the conduit 50 can be attached to any portion of the aorta, such as to a location between the aortic arch 164 and the patient's diaphragm. In certain circumstances (e.g., for extreme cases of calcification of the aorta), the end 56 of the conduit 50 can be attached to the patient's descending aorta 106 at a location that is below the patient's diaphragm. The particular attachment site for the end 56 will vary from patient to patient and may be determined by the surgeon prior to or at the time of the procedure.

FIG. 7 depicts the completed extra-anatomic aortic valve placement in which the end 56 has been anastomosed to the descending aorta 160, such as by sutures 164. Those skilled in the art will understand and appreciate various techniques that can be utilized to appropriately attach the end 56 of the conduit 50 to the patient's aorta as to provide fluid communication from the valve 12, through the conduit 56 and into the aorta. Prior to completing the attachment of the end 56 of the conduit 50 at the descending aorta 160, the seal between the conduit 50 and the valve 12 can be checked. For example, the conduit 50 can be clamped (e.g., by forceps or other clamping instrument) and inflation fluid from the balloon 88 can be removed to determine the efficacy of the attachment of the valve 12 and the conduit 50. Provided that the connection is adequate (e.g., little or no leaking), the catheter 80 can be removed and the conduit can be clamped while the free end 56 is attached to the appropriate implantation site at the descending aorta 160. The balloon 80 can be deflated, such as by removing at least a substantial portion of the inflation fluid using the inflation inlet member 90. After sufficient inflation fluid has been removed from the balloon 88 and the tubular member 82, the catheter 80 can be removed from the patient's heart 102, such as by pulling it through the valve 12 and the interior of the conduit 50. After the catheter has been removed, the remaining portion of the conduit 52 can be attached to the descending aorta 160, as mentioned herein, while the conduit remains clamped.

In view of the foregoing, those skilled in the art will understand and appreciate that the extra-anatomic aortic valve placement provides a low-invasive alternative to aortic valve replacement, such as for patients that might be considered high risk. Additionally, the approach described herein further enables the procedure to be implemented in the absence of cardio pulmonary bypass. It is to be further understood that the patient's aortic valve 116 can be closed, such as by sutures, at some point during the procedure. Alternatively, the aortic valve 116 can remain unmodified, as the procedure may be performed when the aortic valve is substantially non-functioning or dysfunctional. The valve 12 and conduit 50 thus can provide an alternative blood flow path with significantly improved functionality relative to the patient's existing aortic valve 116. The patient's existing valve 116 can be the patient's own native valve or a prior replacement valve, which has failed or no longer functions adequately for the patient.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A method for extra-anatomic aortic valve placement, comprising:
creating an aperture through the patient's heart that extends from a location outside of the patient's heart and into a left ventricle of the heart;
mounting a valve at least partially within the aperture; and
providing a continuous path for fluid communication from the mounted valve and into the patient's aorta.

2. The method of claim 1, further comprising obstructing the aperture to inhibit blood loss through the aperture during at least the mounting of the valve.

3. The method of claim 2, wherein the obstructing of the aperture further comprises:
inserting a balloon catheter into the aperture such that a balloon thereof resides within the left ventricle; and
inflating the balloon such that a proximal portion of the inflated balloon engages a ventricular peripheral portion of the aperture to substantially seal the aperture.

4. The method of claim 3, wherein the balloon catheter comprises a Foley catheter.

5. The method of claim 1, further comprising attaching a sheet of a substantially biocompatible material to the heart in overlying relation to the aperture, the aperture extending through the sheet.

6. The method of claim 5, wherein the sheet comprises a natural tissue material.

7. The method of claim 5, wherein the aperture that extends through the sheet is created substantially concurrently with the creation of the aperture through the patient's heart.

8. The method of claim 1, wherein the providing the continuous path further comprises attaching a length of a flexible conduit between the valve and the aorta.

9. The method of claim 8, wherein the valve further comprises a generally cylindrical sidewall portion that extends between an inflow portion and an outflow portion, at least some of the outflow portion extending outwardly from an exterior surface of the patient's heart after the mounting of the valve within the aperture, the flexible conduit being attached between the outflow portion of the valve and the patient's descending aorta.

10. The method of claim 8, wherein the flexible conduit comprises a natural tissue material.

11. The method of claim 10, wherein the natural tissue material comprises animal pericardium.

12. The method of claim 1, wherein the mounting of the valve further comprises:
providing an implanter having a barrel that contains the valve configured in reduced cross-sectional dimension relative to an expanded cross-sectional dimension of the valve; discharging the valve from the barrel of the implanter into the aperture of the patient's heart; and
expanding the valve within the aperture from the reduced cross-sectional dimension toward the expanded cross-sectional dimension, such that an exterior portion of the valve engages adjacent tissue of the patient's heart to mitigate movement of the valve relative to the adjacent tissue.

13. The method of claim 12, wherein the valve expands automatically in response to being discharged from the barrel of the implanter.

14. The method of claim 12, wherein the valve further comprises a generally cylindrical sidewall portion that extends between an inflow end portion and an outflow portion, at least some of the outflow portion extending outwardly from the patient's heart after the expanding of the valve.

15. The method of claim 14, wherein the valve further comprises a plurality of elongated support features extending generally axially between the opposed ends of the support, the support features being interconnected at ends of the support, and a tissue valve mounted within the support so as to expand radially commensurately with the support in response to the expanding of the valve.

16. The method of claim 15, wherein the valve further comprises a flange that extends radially outwardly from an exterior surface of the outflow portion of the valve, the flange being connected to the patient's heart.

17. The method of claim 12, wherein prior to the mounting of the valve, the method further comprising:
inserting a balloon catheter into the aperture such that a balloon thereof resides within the left ventricle; and
inflating the balloon such that a proximal portion of the inflated balloon engages a ventricular peripheral portion of the aperture to substantially seal the aperture and mitigate blood loss through the aperture during at least the mounting of the valve, an elongated tubular member of the catheter extending through the valve and through the implanter.

18. The method of claim 1, occurring in the absence of cardiopulmonary bypass.

19. The method of claim 1, wherein the aperture is created through the apex of the patient's heart.

20. A method for extra-anatomic aortic valve placement comprising:
creating an aperture through the patient's heart that extends from a location outside of the patient's heart and into a left ventricle of the heart;
obstructing the aperture to inhibit blood loss through the aperture;
mounting a valve at least partially within the aperture while the aperture is obstructed; and
attaching a length of a flexible conduit between an outflow end of the valve and the patient's aorta to provide for substantially unidirectional flow of blood from the left ventricle, through the conduit and into the patient's aorta, the method being performed in the absence of cardio pulmonary bypass.

21. The method of claim 20, wherein the obstructing is performed using a balloon catheter having an elongated tubular member, wherein the mounting further comprises:
providing an implanter having a barrel that contains the valve configured in reduced cross-sectional dimension relative to an expanded cross-sectional dimension of the valve;
discharging the valve from the barrel of the implanter into the aperture of the patient's heart while the tubular member of the catheter extends through the valve and at least partially through the implanter; and
expanding the valve within the aperture from the reduced cross-sectional dimension toward the expanded cross-sectional dimension, such that an exterior portion of the valve engages adjacent tissue of the patient's heart to mitigate movement of the valve relative to the adjacent tissue.

22. A system for extra-anatomic valve placement, the system comprising:
means for creating an aperture through the patient's heart that extends from a location outside of the patient's heart and into a left ventricle of the heart;
means for obstructing the aperture to inhibit blood loss through the aperture;
valve means for, when mounted at least partially within the aperture, providing substantially unidirectional flow of blood from the left ventricle and through the means for providing; and
means for, when connected between the valve means and the patient's aorta, providing for fluid communication along a continuous path from the left ventricle into the patient's aorta.
